(19) United States
(12) Patent Application Publication
(10) Pub. No.: US 2022/0238193 A1
(43) Pub. Date: Jul. 28, 2022

(54) METHODS AND SYSTEMS FOR MANAGING PATIENT-CENTRIC INFORMATION

(71) Applicant: Prognos Health Inc., New York, NY (US)

(72) Inventors: Ali Koc, New York, NY (US); Kristian Kaufmann, New York, NY (US)

(21) Appl. No.: 17/158,906

(22) Filed: Jan. 26, 2021

(51) Int. Cl. G16H 10/60; G16H 15/00; G06F 16/338; G06F 16/33

(52) U.S. Cl. CPC G16H 10/60; G16H 15/00; G16H 50/70; G06F 16/3344; G06F 16/338

(57) ABSTRACT

An in-memory system may be used for storing, partitioning, and analyzing patient information (e.g., lab data/information, healthcare data/information, medical data/information, pharmacy data/information, etc.).

### BACKGROUND

Due to the unique nature of healthcare data, representing the data using conventional data technologies is challenging. General-purpose features associated with conventional data technologies may include, for example, data partitioning. However, do the complex and relationship-dependent nature of patient information (e.g., lab data/information, healthcare data/information, medical data/information, pharmacy data/information, etc.), conventional data technologies are not suitable for partitioning and/or analysis of patient information. Query languages, such as structured query language (SQL) and/or the like ignore distinct properties of the patient-centric data/information, do not enable the construction of healthcare queries and/or questions. Traditional query systems are unable to provide adequate responses to patient-centric queries.

### SUMMARY

It is to be understood that both the following general description and the following detailed description are exemplary and explanatory only and are not restrictive. Methods and systems for managing patient information are described.

A method is described comprising receiving patient query information, wherein the patient query information comprises one or more predicates and one or more patient-specific elements; determining a numeric representation of the one or more predicates and the one or more patient-specific elements; determining, based on the numeric representation of the one or more predicates and an in-memory template, patient information, wherein the in-memory template comprises a numeric representation of patient information associated with a previous request for patient information; retrieving, from one or more nodes of an in-memory cache, based on the patient information, additional patient information, wherein the additional patient information is associated with the one or more patient-specific elements, and causing output of the patient information and the additional patient information.

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of this specification, together with the description, serve to explain the principles of the methods and systems:

FIG. 1 shows an example system;

FIG. 2 shows an example data structure;

FIG. 3 shows an example data structure;

FIG. 4 shows a flowchart of an example method;

FIGS. 5A and 5B show example outputs;

FIG. 6 shows a flowchart of an example method; and

FIG. 7 shows a block diagram of an example computing device.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general-purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

An in-memory system may be used for storing, partitioning, and analyzing patient healthcare data. The system may implement raw data concurrency without synchronization to enable the history (e.g., diagnostic history, dosage history, testing/treatment history, data record, etc.) of a patient to be accessed without a scan (e.g., zero look-up). The system may be patient-centric, enabling all information related to a patient to be stored in one continuous block in memory, for example, associated with an identifier (e.g., a patient ID, etc.).

A patient query and/or request for patient information (e.g., lab data/information, healthcare data/information, medical data/information, pharmacy data/information, etc.) may be analyzed and a population may be determined from the data records. Cached populations responsive to the query may be kept in memory efficiently using roaring bitmaps. An initial population may be determined from bitmaps and used for a full data record query. The system may determine queries that are running on the same population and may use a population that was identified as part of a previous query for future queries. Aggregations and transformations may be performed based on different dimensions of a determined population. Aggregations and transformations may be performed, for example, multiple times, on data records of determined populations to determine a response/result to the query.

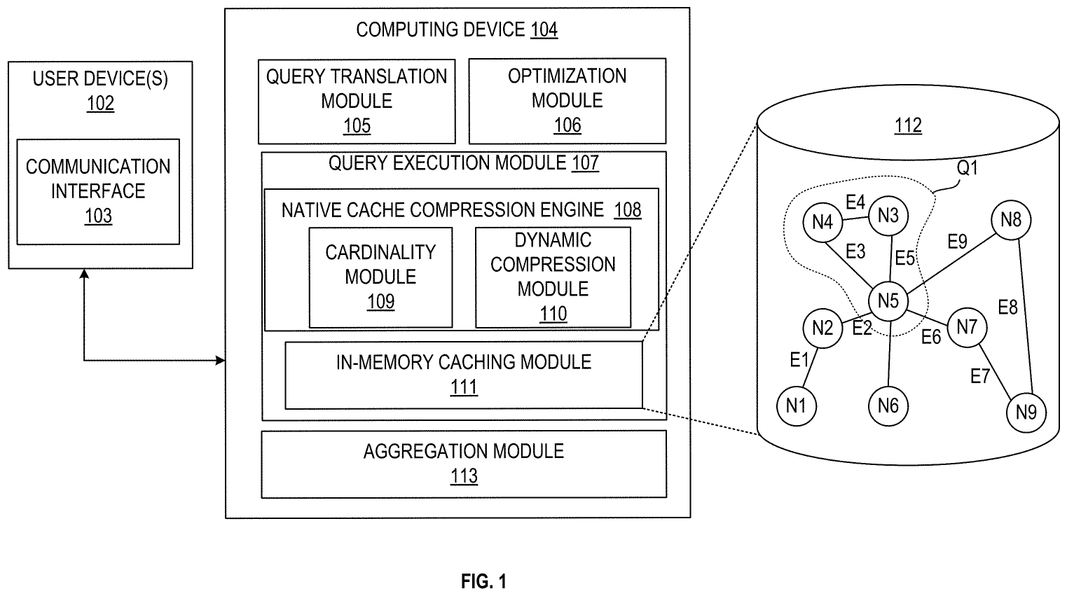

FIG. 1 shows a system 100 for managing patient-centric information. The system 100 enables queries, such as queries relating to patient information (e.g., lab data/information, healthcare data/information, medical data/information, pharmacy data/information, etc.) to be parallelized to increase the speed of execution and/or response. The system 100 enables analysis of a single patient's information and/or history without dependency on that of another patient. The system 100 implements raw concurrency without a synchronization mechanism to hinder performance. For example, the history (e.g., historic data/information, etc.) of a patient may be accessed without a scan (e.g., the system 100 may perform zero lookup queries). The system 100 enables information related to a patient to be stored, for example, in a continuous block in memory under and/or associated with a patient identifier to provide a pure patient-centric storage mechanism. The system 100 utilizes logical structures and processes, such as in-memory templates and cache access algorithms, to improve the way patient-centric data/information is stored and retrieved.

The system 100 may include a user device 102 (e.g., a client device, a smart device, a mobile device, a computing device, etc.). The user device 102 may include a communication interface 103. The communication interface 103 may be and/or include any interface for presenting and/or receiving information to/from a user, such as user feedback. The communication interface 103 may include an interface such as a web browser (e.g., Internet Explorer®, Mozilla Firefox®, Google Chrome®, Safari®, or the like). The communication interface 103 may include physical (e.g., keyboards, mouse, display, touchscreen, etc.) and/or interactive elements (e.g., on-screen buttons/controls, etc.) that enable interactions, actions, functions, and/or the like such as viewing, selecting, manipulating, and/or the like data/information such as patient information (e.g., lab data/information, healthcare data/information, medical data/information, pharmacy data/information, etc.), and/or the like. The communication interface 103 may include any software, hardware, and/or interfaces that enable communication between the user device 102 and a computing device 104 (e.g., a cloud device, a server, a network device, a computer, a query analysis device, data management device, etc.).

The user device 102 (e.g., via the communication interface 103, etc.) may send, request, and/or query information from a local source and/or a remote source, such as the computing device 104. The computing device may store, for example, in-memory, patient information (e.g., lab data/information, healthcare data/information, medical data/information, pharmacy data/information, etc.), such as data/information associated with a plurality (e.g., millions, etc.) of patients. The patient information may be stored as a plurality of data sets. The plurality of data sets may indicate populations of patients (e.g., individuals, users, etc.) that correspond to various categories, classifications, groups, and/or the like used for healthcare analysis.

The user device 102 may send, request, and/or query information from the computing device 104 via and/or based on an application, an API interface, and/or the like. The user device 102 (e.g., via the communication interface 103, etc.) may send, request, and/or query information from the computing device 104 and/or any other device/component of the system 100 via a long-range communication technique (e.g., Internet (HTTP and/or HTTPS request/communications), cellular, satellite, and the like), via a short-range communication technique (e.g., BLUETOOTH®, ZigBee, Z-wave, near-field communication, infrared, etc.), and/or via any communication technique. The user device 102 may, in some scenarios, require authorization and/or authentication to send, request, and/or query information from the computing device 104. The user device 102 may send the computing device 104 an access token to request authorization/authentication and/or authorize/authenticate the user device 102 to send, request, and/or query information from the computing device 104. Authorizing a user device 102 to send, request, and/or query information from the computing device 104 may be based on any method.

The user device 102 may, for example, send and/or request patient-centric information (or any other type of data/information) from the computing device 104. For example, the user device 102 (e.g., via the communication interface 103, etc.) may send the following query (Q1) to the computing device 104:
Q1=“All patients who were diagnosed with X within the last 12 months, started on drug Y within 60 days of diagnosis, and did not change their regimen since that time.”

The query (Q1) may be represented, for example, based on a novel query language used by the system 100. The query language enables queries, for example, healthcare questions, to easily be represented and efficiently executed. The user device 102 may send the query (Q1) or any other query to the computing device 104.

The computing device 104 may receive a query, analyze the query, and determine a response. For example, the computing device 104 may include a query translation module 105. The query translation module 105 may receive the query (Q1) represented by the query language. The use and/or determination of “predicates,” for example, by the computing device 104, enables complex filter expressions to be used to querying resources, such as resources associated with the computing device 104. Q1 may be represented as follows:

```
{
  “predicate”: “SEQUENCE”, “contains”:[
  {“predicate”: “ICD10”, “contains”:[“X”],
  “start-date”: “2019-01-01”, “end-date”: “2019-12-31”},
  {“predicate”: “RX”, “contains”:[“Y”],
  “time-relation”: “before”, “time-days”:60},
  {“predicate”: “NOT”, “predicates”:[
  {“predicate”: “RX”, “contains”:[“*”],
  “time-relation”: “before”, “time-days”:3650}]}]
}
```

The determination and/or use of predicates enables complex filter expressions when querying resources. For example, query predicates may be translated into resource and/or database queries. Within a sequence query, predicates may be time-constrained (e.g., “time-relation”) relative to the occurrence of previous events. For example:
Within: Two events occurring within X days of each other where order does not matter;
Before: Event must occur within X days after the previous event;
After: Event must occur at least X days after the previous event.

Each predicate in a query can be constrained with specific absolute date ranges to enable the computing device 104 to determine/identify events within known dates. The computing device 100 may be used to analyze a set of events within a query that are occurring relative to an index event. Event queries enable an index event to be defined along with a set of must-have and must-not-have events, each of which is constrained to the index event with relative days where the index event is assumed to be day zero. For example, Q1 may be represented as:

```
{
    “predicate”: “EVENT”, “contains”:[
  { “predicate”: “ICD10”, “contains”:[ “X”],
    “start-date”: “2019-01-01”, “end-date”: “2019-12-31”},
  { “predicate”: “MUST-NOT-HAVE”, “predicates”:
  [{ “predicate”: “ICD10”, “contains”:[ “X”]}],
  “start-day”:−3650, “end-day”:−1}
  { “predicate”: “MUST-HAVE”, “predicates”:
    [{ “predicate”: “RX”, “contains”:[ “Y”]}],
    “start-day”:0, “end-day”:60}
  ]
}
```

The computing device 104, for example, via the query translation module 105, may translate a received query to a numeric equivalent, such as integer values. The query translation module 105 may determine a numeric representation of query predicates and patient-specific data/information included with a query. A numeric representation of a query may be used to determine/identify a subpopulation of patients that correspond to the query. The computing device 104 may determine the arrangement and/or pattern of predicates in the query to determine a subpopulation of patients that correspond to the query (e.g., a result to the query, etc.). For example, based on an in-memory template, such as a bitmap (e.g., roaring bitmaps, etc.), and an in-memory data store (e.g., an undirected graph database, etc.). The in-memory template is a specific type of data structure designed to improve the way data/information is stored and retrieved, for example, in memory.

Each type of query predicate may require a different amount of execution resources available to the computing device 104. To manage the resources available to the computing device 104, the computing device 100 may include a query optimizer 106. The query optimizer 106 may arrange predicates in a process order to maximize the efficiency of query execution. For example, a predicate related to the gender of a patient may require fewer resources and/or execute faster than a predicate used to check for the existence of data/information, such as a patient record, within a specified date range. Filtering the patient based on gender may require a single lookup and/or comparison, while the existence of a record and/or event within a given time period may require going over the event history of and/or associated with the patient.

The query optimizer 106 may use short-circuit evaluation (e.g., minimal evaluation) on AND logic to increase the processing speed of queries. For example, based on a first argument, a second argument may be executed and/or evaluated only if the first argument does not suffice to determine the value of an expression. When, for example, the first argument of an AND function evaluates to false, the overall value must be false. The computing device 104, for example, when determining predicates inside an AND logic gate, may first find a predicate that evaluates to false and may stop evaluating the rest of the predicates in that logic. The query optimizer 106 may determine/identify predicates inside AND logic and order the predicates that may be executed faster as first in the query.

For example, the system 100 (e.g., the query optimizer 106, etc.) may assign a weight to each argument of the query. Arguments that may be evaluated/processed rapidly, easily, and/or the like may be associated with a lower weight, and weights for each argument may increase according to the degree/amount of evaluation/processing time associated with the argument. For example, for a query for all patients over the age of 60 who have had a particular lab test in their history, the query may be constructed as follows:

### HAD LAB TEST X AND ABOVE THE AGE OF 60

Notably, It is much faster to include/exclude a patient based on his/her age than to determine from the data records (e.g., scan through the records) of the patient whether they had the particular test. The query may be rewritten as follows:

### ABOVE THE AGE OF 60 AND HAD LAB TEST X.

The query optimizer 106 may evaluate the first argument (above the age of 60) and if false, skip the second argument (had lab test X).

A query that has been translated to a numeric representation with predicates that have been ordered for efficiency, may be executed to determine a result (e.g., a responsive population, etc.). The computing device 104 may include a query execution module 107. The query execution module 107 may determine and/or detect queries that are running on the same population, for example, queries received from multiple user devices (e.g., the user device(s) 102, etc.), repeat queries, and/or the like, and use a population determined from a previous query for current and/or future queries. The computing device 104 may cache populations in memory using an in-memory template and/or data structure, such as a roaring bitmap and/or the like to expedite the execution of a query.

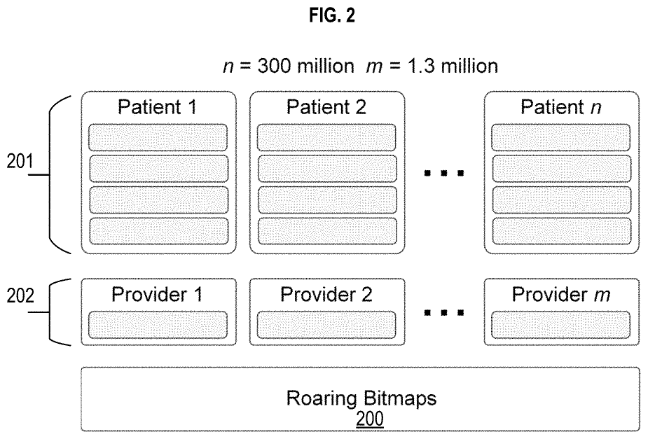

FIG. 2 is an example block diagram of an in-memory cache of the computing device 104. Roaring bitmaps 200 may be used to correlate a plurality (e.g., 300 million+, etc.) of patient data sets 201 and a plurality (e.g., 1.3 million+, etc.) of provider (e.g., healthcare provider, health care provider, etc.) data sets 202.

Returning to FIG. 1, the computing device 104 may store major fields of patient information/data as roaring bitmaps. For example, computing device 104 may store bitmaps on de-identified data (TABLE 1), mapping various aspects of the data to patients:

TABLE 1

| FIELD TYPES | FIELDS (Indicies) |
|---|---|
| Demographics | Gender, State, Birth Year |
| Lab Tests | Test Type, ICD |
| Rx/Medical Claims | Drug Name, ICD |
| Providers | NPI, Provider Specialty |

Each field may specify an integer size. For example, fields like gender and state may be represented using unsigned 8-bit integers. Larger cardinality fields such as provider NPI may be represented as unsigned 32-bit integers. Dates may also be translated to “number of days since YEAR XYZ” and kept as unsigned 16-bit integers.

In-memory templates, such as roaring bitmaps, enable the computing device 104 to quickly determine patients who have a certain test, drug, and/or diagnosis in their history without accessing corresponding patient information (e.g., patient history, etc.). A query received by the computing device 104 may be initially analyzed, for example, based on bitmap (or any other in-memory template and/or data structure) operations that ignore certain aspects of the query such as date constraints, test values, and drug days-supply. The computing device 104 may break up integers determined by the query translation module 105 into buckets/containers of 2¹⁶ integers (65535). The in-memory template and/or roaring bitmap architecture provides a key-value structure where each key-value pair represents a set (S) of all 32-bit integers that share the same most significant 16 bits. The key is made of the shared 16 bits, whereas the value is a container storing the remaining 16 least significant bits for each member of the set (S) (e.g., partitioned as 2¹⁶ spaces). when implemented as arrays, an array may be used to store keys and an array may be used to store values. Forming a simple in-memory (RAM) key-value data store.

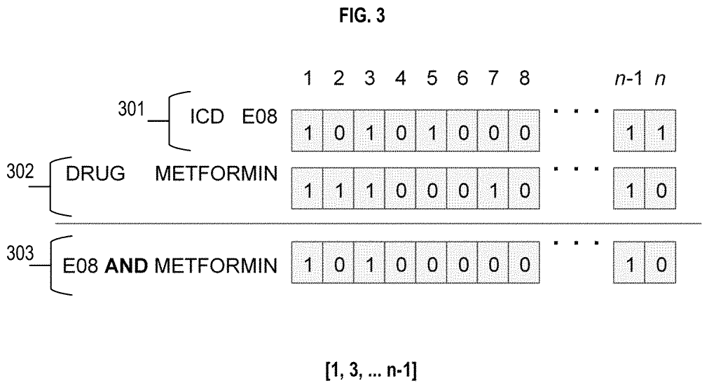

FIG. 3 shows an example of a bitmap of patient information. FIG. 3. shows a container 301 of integers corresponding to patients diagnosed with Diabetes mellitus, as indicated by the International Statistical Classification of Diseases and Related Health Problems (ICD) for a specific disease code E08. Container 302 includes integers corresponding to patients treated with the drug Metformin, and container 303 includes integers corresponding to patients diagnosed with Diabetes mellitus and treated with the drug Metformin.

Returning to FIG. 1, the native query execution module 107 may include a native cache compression engine 108 and an in-memory caching module 111. The native cache compression engine 108 may execute the query, for example, through pure bitmap operations and/or the like. The in-memory caching module 111 may execute the query, for example, based on a complete data record, such as full patient history.

The native cache compression engine 108 may ignore certain aspects of the query such as date constraints, test values, and drug days-supply. The native cache compression engine 108 may access data values from the cache of the computing device 104 and implement a compression algorithm (e.g., using roaring bitmaps, etc.) based on a compression rate derived from a cardinality of the data values. The native cache compression engine 108 may determine/generate an elastic or dynamically compression-adjusted local cache.

The native cache compression engine 108 may include a cardinality module 109 and a dynamic compression module 110. The cardinality module 109 may be used to determine the cardinality of the data values in the cache. The cardinality may refer to and/or indicate the uniqueness of data values and/or entropy of data values, contained in a particular attribute of a container and/or data table. In some instances, cardinality may refer to and/or indicate data values to be compressed. The higher the cardinality, the more duplicated elements in an array column.

The dynamic compression module 110 may determine, for example, based on the cardinality of the data values in the cache, a compression ratio. The dynamic compression module 110 may set attributes for compression, such as the number of roaring bitmaps to allocate, based on the results. The dynamic compression module 110 may take other actions based on the results of the cardinality analysis, such as determine a different compression algorithm. For example, the dynamic compression module 110 may cause the compression ratio to be proportional to the cardinality of the data values. Query evaluation is most efficient when both the number of bitmaps and the size of each bitmap are small in size. The dynamic compression module 110 may determine bitmaps with the smallest sizes possible.

When a query, for example, Q1, is received the query execution module may access a field and/or index (e.g., determined from one or more predicates), determine corresponding bitmaps for related values, perform bitwise logical AND operations to get a new bitmap and/or access the corresponding stored data. The corresponding data may, for example, indicate and/or be associated with an initial population and/or subpopulation of patients. For example, an initial population such as a population that includes all patients associated with a provider or all providers associated with a patient may be determined based on roaring bitmaps without data scanning. The system 100 enables the computing device 104 to operate as an undirected graph database, where the dimensions of the data are connected through patient nodes (e.g., patient data sets 201, etc.) and the relationship between a particular data point, such as a lab test, and a population, such as a plurality of healthcare providers, may be determined without scanning the data.

A full patient history of patients (e.g., populations, etc.) determined from bitmap operations may be used to determine a final population. A final population may be cached for future queries and/or used for further analysis (e.g., aggregation, etc.). The computing device may include an in-memory caching module 111 that caches and/or stores populations for future queries and/or used for further analysis.

The in-memory caching module 111 may include, for example, a graph database 112. The graph database 112 may include a plurality of nodes N1-N9, each containing one or more data items (e.g., patient information, etc.) and/or properties describing the data item (e.g., fields, indices, etc.). Each of the nodes N1-N9 may be associated with a node identifier and one or more data items and/or properties. The nodes N1-N9 may be interconnected via a plurality of edges E1-E9 describing the relationships between the nodes N1-N9. Each of the edges E1-E9 may be associated with an identifier and an edge group/type. The edges E1-E9 may include additional information based on relationships between the nodes N1-N9.

A query, such as Q1, to the graph database 112 may be represented as structured of nodes N4-N5 and edges E3-E5. When executing Q1 a sub-graph within the graph database 112 that corresponds to (e.g., is isomorphic to, includes the pattern indicated by the structure of Q1 is determined. Determining the structure of Q1 (e.g., N3-N3 and E3-E5) may include a Boolean match, a target node match, or a complete structure match. For example, a Boolean match, a result (e.g., a full patient history, etc.) may produce a Boolean indication of a match or lack/absence thereof. Executing Q1 based on a target query node may include determining a match of a complete query sub-graph, but only returning/responding with one or more of the nodes of the query (e.g., N3) within the graph database 112. Executing Q1 based on a complete match may include determining a match of the complete structure of Q1 against the graph database 112.

FIG. 1 shows a simple representation of the data store 112. The data store 112 may include any number of nodes and/or edges. In some instances, when the computing device 104 receives a query, a plurality of subgraphs (e.g., a group of nodes and/or edges, subpopulations, etc.) that correspond to the query. A subgraph may correspond to patient information and/or records for a specific patient or a plurality of patients. A subgraph may correspond to patient information and/or records for a determined subpopulation. The computing device 104 may distribute the subgraphs to a plurality of slave processors (not shown). The slave processors may be native to the computing device and/or distributed among one or more computing devices. The slave processors may, for example, enable parallelization of query analysis. For example, the slave processors may simultaneously (in parallel) process subgraphs to determine one or more data/information matches of/for the query.

Parallelization may be executed and/or performed in a patient-centric manner. For example, a total number of patients for which data/information is associated with and/or available within the system 100 may be divided equally among a number of processor. For example, a 4-computing-core device (e.g., a node) may manage and/or be associated with patient-centric data/information for eight (8) patients. Patients (patient data/information) may be split into four (4) groups, for example, two (2) patients may correspond to each group. Each group may be processed by a processor (e.g., a slave processor, etc). The describe parallelization enables quick/rapid evaluation of each patient without the need to cross-thread synchronization between computing cores. A query may be parallelized (e.g., arranged and executed as a plurality of subgraphs, etc.), executed for a determined subpopulation, and cached for future aggregation or analytics requests.

The computing device 104 may include an aggregation module 113. The aggregation module 113 may aggregate subgraph results. The aggregation module 113 may aggregate data/information and/or records of determined/patients based on aggregated subgraphs.

The computing device 104 may include an authorization and authentication control (AAC) module 114. Data/information, such as patient information, may be associated with access rights. When the computing device 104 receives a query request, the AAC module 114 may ensure that a requester, such as a user device 102, is only allowed to access records the requester is authorized to access. The AAC module 114 may include and/or communicate with one or more access control service, for example, via a webhook, to determine if a user device 102 is authorized to access patient information. Authorization and/or authentication to access patient information may be based on dates associated with a data record, tags defined on the data record, and/or a use case associated with a request. The computing device 104 may store, manage, and/or track patient information, provider information, and any associated relationship without the overhead from access control.

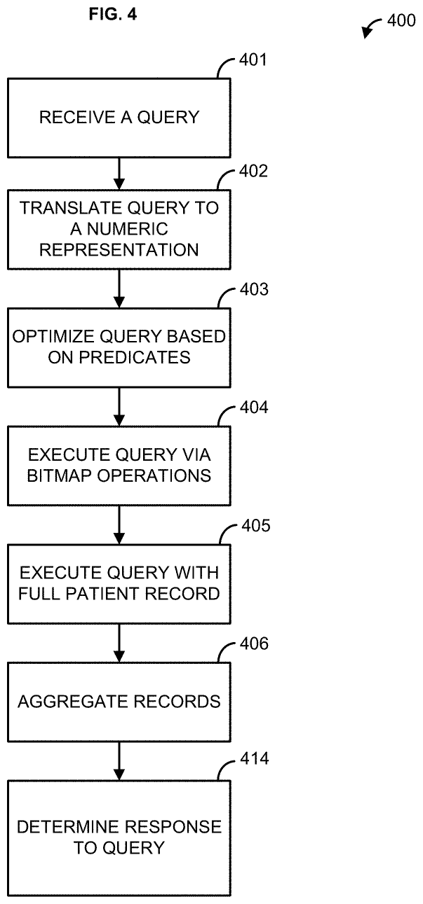

FIG. 4 is an example flowchart of a method 400 for managing patient-centric information. At 401, a query may be received. The query may be a request for information, such as a request for patient information (e.g., lab data/information, healthcare data/information, medical data/information, pharmacy data/information, etc.). A user device (e.g., a client device, a smart device, a mobile device, the user device 102, a computing device, etc.) may send the query to a computing device (e.g., a cloud device, a server, a network device, a computer, a query analysis device, data management device, the computing device 104, etc.).

At 402, the query may be translated. For example, a numeric representation of the query may be determined. For example, the computing device may translate query identifiers to a numeric/integer equivalent and/or representation. The query identifiers may be any representation within a language of items associated with the query. For example, a query may include a request for “All patients who were diagnosed with X within the last 12 months, started on drug Y within 60 days of diagnosis, and did not change their regimen since that time.” Machine learning and/or natural language processing may be used to determine corresponding query identifiers. Corresponding query identifiers may include representations of predicates, dimensions (e.g., temporal dimensions, time constraints/relations, etc.), and/or the like.

At 403, the query may be optimized. For example, the computing device may order/arrange predicates for efficient query execution. For example, a predicate related to the gender of a patient may require fewer resources and/or execute faster than a predicate used to check for the existence of data/information, such as a patient record, within a specified date range. Filtering the patient based on gender may require a single lookup and/or comparison, while the existence of a record and/or event within a given time period may require going over the event history of and/or associated with the patient.

At 403, the query may be executed through pure bitmap operations. The computing device may execute the query using pure bitmap (e.g., roaring bitmap, etc.) operations to determine/identify an initial population responsive to the query, such as an initial population of patients responsive to a patient-centric query.

At 404, the query may be executed based on full patient history and/or data record. The computing device may access, for example, an in-memory graph data store to determine one or more nodes and/or edges associated with and/or responsive to the query based on the determined initial population. The initial population may be used to determine one or more root nodes of the graph data store. Edges of each root node may be simultaneously processed/executed (e.g., parallel processing, etc.) to determine additional nodes and/or edges responsive to the query, such as one or more nodes and edges that store/include a full patient history and/or data record. The one or more nodes and/or edges associated with and/or responsive to the query may indicate and/or be used to determine, for example, a second population (e.g., a subgroup, etc.) of patients responsive to the query. The second population may be smaller than the initial population determined through pure bitmap operations.

At 405, data records from the full patient history may be aggregated. The computing device may analyze and aggregate data records responsive to the query determined from the second population. The aggregated data records may indicate and/or be associated a third population responsive to the query. The third population may be and/or indicate a response to the query.

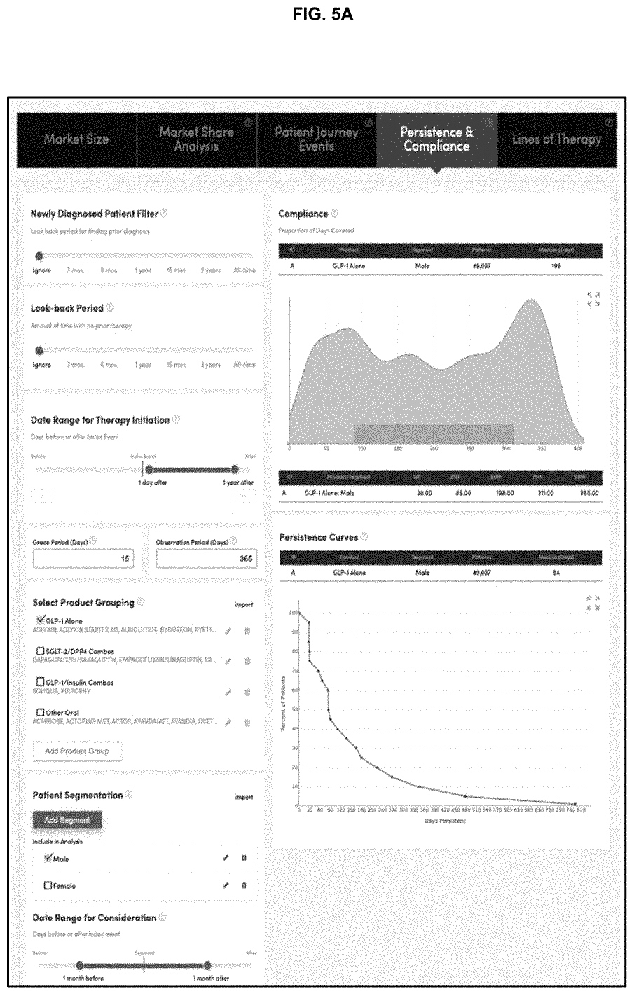

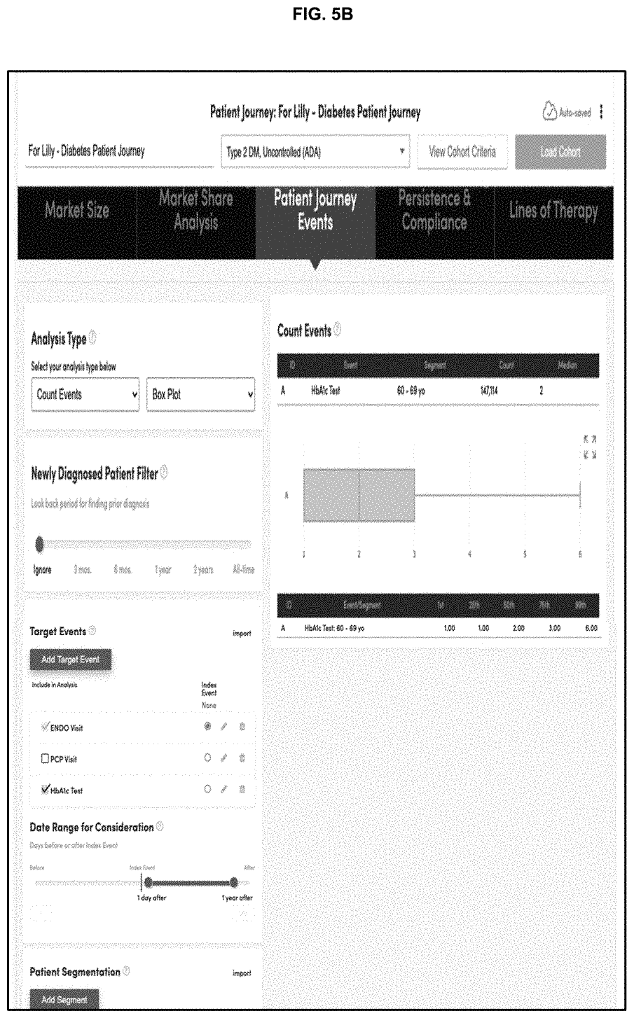

At 406, a response to the query may be determined. The computing device may output the response. The computing device may translate numeric identifiers of the third population to text values. The computing device may use the text values to generate an audible response to the query, a graphical response to the query, a textual response to the query, combinations thereof, and/or the like. The computing device may cause a display of the response (and/or a portion of the response) to the query. The computing device may send the response (and/or a portion of the response) to the user device. FIGS. 5A and 5B show example displays of responses to a query. FIG. 5A shows the response to a sequence-based query. FIG. 5A shows the response to an event-based query.

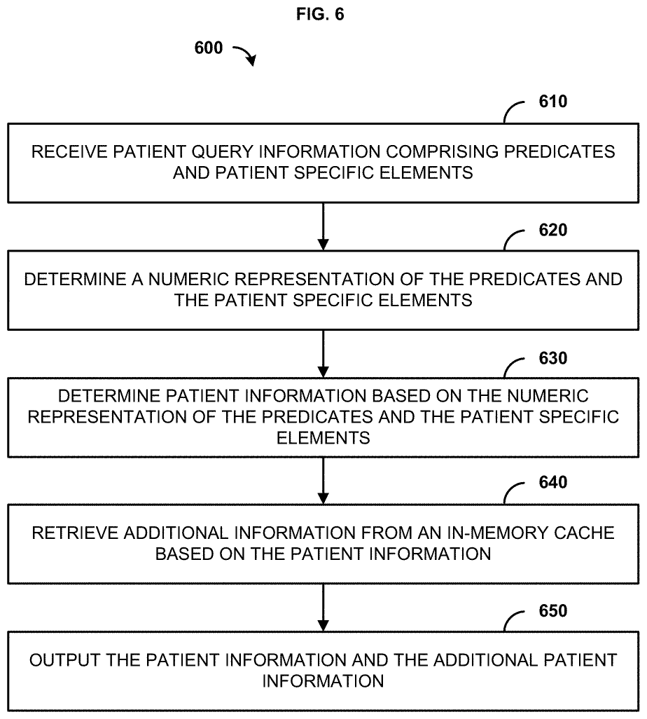

FIG. 6 is an example flowchart of a method 600 for managing patient-centric information. At 610, receiving patient query information. The patient query information may include one or more predicates and one or more patient-specific elements. Receiving the patient query information may include receiving the patient query information from a user device. The patient query information may include natural language query information.

At 620, determining a numeric representation of the one or more predicates and the one or more patient-specific elements.

At 630, determining, based on the numeric representation of the one or more predicates and an in-memory template, patient information. The in-memory template may include a numeric representation of patient information associated with a previous request for patient information. The in-memory template may be and/or include, for example, a roaring bitmap. The in-memory template may be and/or include any data structure.

Determining the patient information may include: mapping, based on an order associated with an estimated process time for each predicate element of the one or more predicates, one or more values of the numeric representation of the one or more predicates to one or more values of the numeric representation of the patient information associated with the previous request, and determining, based on the mapping between the one or more values of the numeric representation of the one or more predicates and the one or more values of the numeric representation of the patient information associated with the previous request for patient information, the patient information.

At 640, retrieving, from one or more nodes of an in-memory cache, based on the patient information, additional patient information. The additional patient information may be associated with the one or more patient-specific elements. Retrieving the additional patient information may include: determining that the one or more patient-specific elements correspond to the one or more nodes, and determining, based on the one or more nodes and the patient information, one or more additional nodes of the in-memory cache, wherein the additional nodes comprise the additional patient information.

At 650, causing output of the patient information and the additional patient information. causing the output of the patient information and the additional patient information may include causing a user device to display the patient information and the additional patient information. The output of the patient information and the additional patient information may include one or more of a textual representation of the patient information and the additional patient information, a graphical representation of the patient information and the additional patient information, or a statistical representation of the patient information and the additional patient information

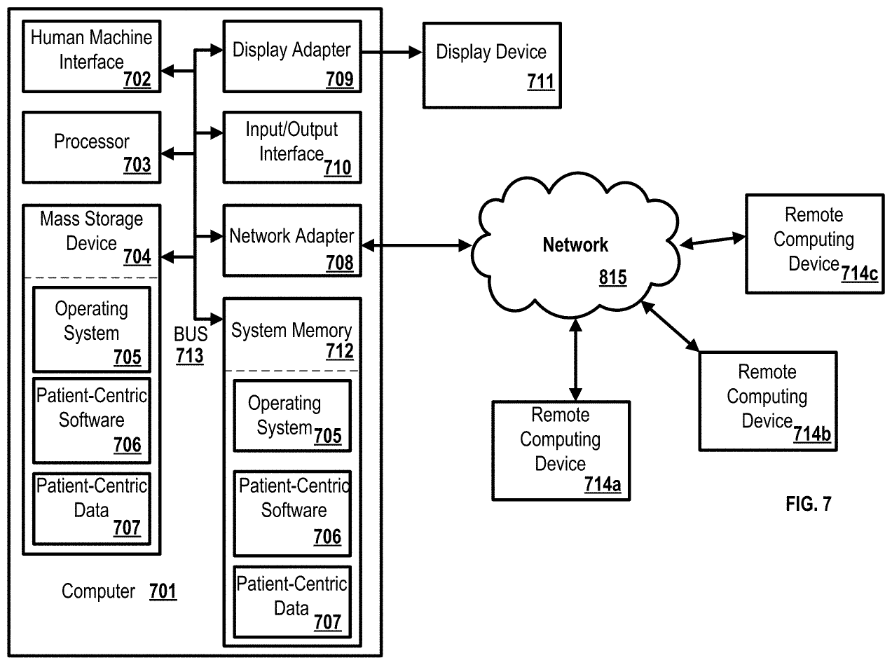

FIG. 7 shows a system 700 for managing patient-centric information. The user device 102, the computing device 104, and/or any other device/component described herein may be a computer 701 as shown in FIG. 7.

The computer 701 may comprise one or more processors 703, a system memory 712, and a bus 713 that couples various components of the computer 701 including the one or more processors 703 to the system memory 712. In the case of multiple processors 703, the computer 701 may utilize parallel computing.

The bus 713 may comprise one or more of several possible types of bus structures, such as a memory bus, memory controller, a peripheral bus, an accelerated graphics port, and a processor or local bus using any of a variety of bus architectures.

The computer 701 may operate on and/or comprise a variety of computer-readable media (e.g., non-transitory). Computer-readable media may be any available media that is accessible by the computer 701 and comprises, non-transitory, volatile and/or non-volatile media, removable and non-removable media. The system memory 712 has computer-readable media in the form of volatile memory, such as random access memory (RAM), and/or non-volatile memory, such as read-only memory (ROM). The system memory 712 may store data such as patient-centric data 707 and/or program modules such as operating system 705 and patient-centric software 706 that are accessible to and/or are operated on by the one or more processors 703.

The computer 701 may also comprise other removable/non-removable, volatile/non-volatile computer storage media. The mass storage device 704 may provide non-volatile storage of computer code, computer-readable instructions, data structures, program modules, and other data for the computer 701. The mass storage device 704 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 704. An operating system 705 and patient-centric software 706 may be stored on the mass storage device 704. One or more of the operating system 705 and patient-centric software 706 (or some combination thereof) may comprise program modules and the patient-centric software 706. Patient-centric data 707 may also be stored on the mass storage device 704. Patient-centric data 707 may be stored in any of one or more databases known in the art. The databases may be centralized or distributed across multiple locations within the network 715.

A user may enter commands and information into the computer 701 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 703 via a human-machine interface 702 that is coupled to the bus 713, but may be connected by other interface and bus structures, such as a parallel port, game port, an IEEE 1394 Port (also known as a Firewire port), a serial port, network adapter 708, and/or a universal serial bus (USB).

A display device 711 may also be connected to the bus 713 via an interface, such as a display adapter 709. It is contemplated that the computer 701 may have more than one display adapter 709 and the computer 701 may have more than one display device 711. A display device 711 may be a monitor, an LCD (Liquid Crystal Display), a light-emitting diode (LED) display, a television, a smart lens, smart glass, and/or a projector. In addition to the display device 711, other output peripheral devices may comprise components such as speakers (not shown) and a printer (not shown) which may be connected to the computer 701 via Input/Output Interface 710. Any step and/or result of the methods may be output (or caused to be output) in any form to an output device. Such output may be any form of visual representation, including, but not limited to, textual, graphical, animation, audio, tactile, and the like. The display 711 and computer 701 may be part of one device, or separate devices.

The computer 701 may operate in a networked environment using logical connections to one or more remote computing devices 714a,b,c. A remote computing device 714a,b,c may be a personal computer, computing station (e.g., workstation), portable computer (e.g., laptop, mobile phone, tablet device), smart device (e.g., smartphone, smartwatch, activity tracker, smart apparel, smart accessory), security and/or monitoring device, a server, a router, a network computer, a peer device, edge device or other common network nodes, and so on. Logical connections between the computer 701 and a remote computing device 714a,b,c may be made via a network 715, such as a local area network (LAN) and/or a general wide area network (WAN). Such network connections may be through a network adapter 708. A network adapter 708 may be implemented in both wired and wireless environments. Such networking environments are conventional and commonplace in dwellings, offices, enterprise-wide computer networks, intranets, and the Internet.

Application programs and other executable program components such as the operating system 705 are shown herein as discrete blocks, although it is recognized that such programs and components may reside at various times in different storage components of the computing device 701, and are executed by the one or more processors 703 of the computer 701. An implementation of patient-centric software 706 may be stored on or sent across some form of computer-readable media. Any of the disclosed methods may be performed by processor-executable instructions embodied on computer-readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
receiving patient query information, wherein the patient query information comprises one or more predicates and one or more patient-specific elements;
determining a numeric representation of the one or more predicates and the one or more patient-specific elements;
determining, based on the numeric representation of the one or more predicates and an in-memory template, patient information, wherein the in-memory template comprises a numeric representation of patient information associated with a previous request for patient information;
retrieving, from one or more nodes of an in-memory cache, based on the patient information, additional patient information, wherein the additional patient information is associated with the one or more patient-specific elements; and
causing output of the patient information and the additional patient information.

2. The method of claim 1, wherein receiving the patient query information comprises receiving the patient query information from a user device.

3. The method of claim 1, wherein the patient query information comprises natural language query information.

4. The method of claim 1, wherein the in-memory template comprises a roaring bitmap.

5. The method of claim 1, wherein determining the patient information comprises:
mapping, based on an order associated with an estimated process time for each predicate element of the one or more predicates, one or more values of the numeric representation of the one or more predicates to one or more values of the numeric representation of the patient information associated with the previous request; and
determining, based on the mapping between the one or more values of the numeric representation of the one or more predicates and the one or more values of the numeric representation of the patient information associated with the previous request for patient information, the patient information.

6. The method of claim 1, wherein retrieving the additional patient information comprises:
determining that the one or more patient-specific elements correspond to the one or more nodes; and
determining, based on the one or more nodes and the patient information, one or more additional nodes of the in-memory cache, wherein the additional nodes comprise the additional patient information.

7. The method of claim 1, wherein causing the output of the patient information and the additional patient information comprises causing a user device to display the patient information and the additional patient information.

8. The method of claim 1, wherein the output of the patient information and the additional patient information comprises one or more of a textual representation of the patient information and the additional patient information, a graphical representation of the patient information and the additional patient information, or a statistical representation of the patient information and the additional patient information.

9. The method of claim 1, wherein receiving the patient query information comprises receiving the patient query information via a user interface.

10. One or more non-transitory computer-readable media storing processor-executable instructions thereon that, when executed by a processor, cause the processor to:
receive patient query information, wherein the patient query information comprises one or more predicates and one or more patient-specific elements;
determine a numeric representation of the one or more predicates and the one or more patient-specific elements;
determine, based on the numeric representation of the one or more predicates and an in-memory template, patient information, wherein the in-memory template comprises a numeric representation of patient information associated with a previous request for patient information;
retrieve, from one or more nodes of an in-memory cache, based on the patient information, additional patient information, wherein the additional patient information is associated with the one or more patient-specific elements; and
cause output of the patient information and the additional patient information.

11. The one or more computer-readable media of claim 10, wherein the processor-executable instructions that cause the at least one processor to receive the patient query information further cause the at least one processor to receive the patient query information from a user device.

12. The one or more computer-readable media of claim 10, wherein the patient query information comprises natural language query information.

13. The one or more computer-readable media of claim 10, wherein the in-memory template comprises a roaring bitmap.

14. The one or more computer-readable media of claim 10, wherein the processor-executable instructions that cause the at least one processor to determine the patient information further cause the at least one processor to:
map, based on an order associated with an estimated process time for each predicate element of the one or more predicates, one or more values of the numeric representation of the one or more predicates to one or more values of the numeric representation of the patient information associated with the previous request; and
determine, based on the mapping between the one or more values of the numeric representation of the one or more predicates and the one or more values of the numeric representation of the patient information associated with the previous request for patient information, the patient information.

15. The one or more computer-readable media of claim 10, wherein the processor-executable instructions that cause the at least one processor to retrieve the additional patient information further cause the at least one processor to:
determine that the one or more patient-specific elements correspond to the one or more nodes; and
determine, based on the one or more nodes and the patient information, one or more additional nodes of the in-memory cache, wherein the additional nodes comprise the additional patient information.

16. The one or more computer-readable media of claim 10, wherein the processor-executable instructions that cause the at least one processor to cause the output of the patient information and the additional patient information further cause the at least one processor to cause a user device to display the patient information and the additional patient information.

17. The one or more computer-readable media of claim 10, wherein the output of the patient information and the additional patient information comprises one or more of a textual representation of the patient information and the additional patient information, a graphical representation of the patient information and the additional patient information, or a statistical representation of the patient information and the additional patient information.

18. The one or more computer-readable media of claim 10, wherein the processor-executable instructions that cause the at least one processor to receive the patient query information cause the at least one processor to receive the patient query information via a user device.

19. An apparatus comprising:
one or more processors; and
a memory storing processor-executable instructions that, when executed by the one or more processors, cause the apparatus to:
receive patient query information, wherein the patient query information comprises one or more predicates and one or more patient-specific elements;
determine a numeric representation of the one or more predicates and the one or more patient-specific elements;
determine, based on the numeric representation of the one or more predicates and an in-memory template, patient information, wherein the in-memory template comprises a numeric representation of patient information associated with a previous request for patient information;
retrieve, from one or more nodes of an in-memory cache, based on the patient information, additional patient information, wherein the additional patient information is associated with the one or more patient-specific elements; and
cause output of the patient information and the additional patient information.

20. The apparatus of claim 19, wherein the in-memory template comprises a roaring bitmap.
